(19) United States
(12) Patent Application Publication
(10) Pub. No.: US 2013/0225627 A1
(43) Pub. Date: Aug. 29, 2013

(54) DEXTROMETHORPHAN ANTITUSSIVE COMPOSITIONS

(71) Applicants: Chandra Ulagaraj Singh, San Antonio, TX (US); Jagaveerabhadra Rao Nulu, Austin, TX (US)

(72) Inventors: Chandra Ulagaraj Singh, San Antonio, TX (US); Jagaveerabhadra Rao Nulu, Austin, TX (US)

(73) Assignee: Trinity Laboratories, Inc., San Antonio (TX)

(21) Appl. No.: 13/813,630

(22) PCT Filed: Aug. 1, 2011
(86) PCT No.: PCT/US11/46157

§ 371 Date: Apr. 8, 2013

Related U.S. Application Data
(60) Provisional application No. 61/369,742, filed on Aug. 1, 2010.

(51) Int. Cl. A61K 31/485

(52) U.S. Cl. CPC A61K 31/485
USPC 514/289

(57) ABSTRACT

The invention provides orally administered antitussive pharmaceutical compositions comprising dextromethorphan, wherein the compositions are free of bromide, sodium and polistirex.

### CROSS REFERENCE TO RELATED APPLICATIONS

This application claims the benefit of U.S. Provisional Appl. No. 61/369,742, filed Aug. 1, 2010. The content of the aforesaid application is relied upon and incorporated by reference in its entirety.

### FIELD OF THE INVENTION

The field of the invention relates generally to antitussive compositions containing dextromethorphan, essentially free of bromide, sodium and polistirex and methods of administering the same to subjects in need thereof.

### BACKGROUND OF THE INVENTION

Dextromethorphan (racemethorphan), 3-methoxy-17-methylmorphinan, is disclosed on p. 1170 in the Merck Index, 10th Edition (1983), M. Windholz, ed., No. 8009, as an antitussive agent.

Dextromethorphan hydrobromide is used extensively as an antitussive agent in commercial products as disclosed in the Physicians' Desk Reference for Nonprescription Drugs, 11th Edition (1990), E. R. Barnhardt, pub., p. 306; and in Physicians' Desk Reference, 44th Edition (1990), E. R. Barnhardt, pub., p. 309, as follows:
Bayer Children's Cough Syrup by Glenbrook, Benylin DM by Parke-Davis, Benylin Expectorant by Parke-Davis, Cerose-DM by Wyeth-Ayerst, Cheracol D Cough Formula by Upjohn, Cheracol Plus Head Cough/Cold Formula by Upjohn, Cough Formula Comtrex by Bristol-Myers Products, Comtrex Multi-Symptom Cold Reliever Tablets/Caplets/Liquid/Liquigels by Bristol-Myers Products, Contac Cough Formula by SmithKline Consumer, Contac Cough & Sore Throat Formula by SmithKline Consumer, Contac Jr. Children's Cold Medicine by SmithKline Consumer, Contac Nighttime Cold Medicine by SmithKline Consumer, Contac Severe Cold Formula Caplets by SmithKline Consumer, Dimacol Caplets by Robins, Dorcol Children's Cough Syrup by Sandoz Consumer, Hold by SmithKline Beecham, Naldecon DX Adult Liquid by Bristol Laboratories, Naldecon DX Children's Syrup by Bristol Laboratories, Naldecon DX Pediatric Drops by Bristol Laboratories, Naldecon Senior DX Cough/Cold Liquid by Bristol Laboratories, Novahistine DMX by Lakeside Pharmaceuticals, Pediacare Cough-Cold Formula Liquid and Chewable Tablets by McNeil Consumer Products, Pediacare Night Rest Cough-Cold Formula Liquid by McNeil Consumer Products, Robitussin Night Relief by Robins, Robitussin-CF by Robins, Robitussin-DM by Robins, Scot-Tussin Sugar-Free DM Cough & Cold Medicine by Scot-Tussin, Snaplets-DM by Baker Cummins Pharmaceuticals, Snaplets-Multi by Baker Cummins Pharmaceuticals, St. Joseph Cough Suppressant for Children by Plough, St. Joseph Nighttime Cold Medicine by Plough, Sucrets Cough Control Formula by SmithKline Beecham, Sudafed Cough Syrup by Burroughs Wellcome, Triaminic Night Light by Sandoz Consumer, Triaminic-DM Syrup by Sandoz Consumer, Triaminicol Multi-Symptom Cold Tablets by Sandoz Consumer, Triaminicol Multi-Symptom Relief by Sandoz Consumer, Tylenol Cold Medication Caplets and Tablets by McNeil Consumer Products, Tylenol Cold Medication Liquid by McNeil Consumer Products, Tylenol Cold Medication No Drowsiness Formula Caplets by McNeil Consumer Products, Vicks Children's Cough Syrup by Richardson-Vicks, Inc., Vicks Children's NyQuil by Richardson-Vicks, Inc., Vicks Cough Silencers Cough Drops by Richardson-Vicks, Inc., Vicks Daycare Daytime Colds Medicine Caplets by Richardson-Vicks, Inc., Vicks Daycare Multi-Symptom Colds Medicine Liquid by Richardson-Vicks, Inc., Vicks Formula 44 Cough Control Discs by Richardson-Vicks, Inc., Vicks Formula 44 Cough Medicine by Richardson-Vicks, Inc., Vicks Formula 44D Decongestant Cough Medicine by Richardson-Vicks, Inc., Vicks Formula 44M Multi-Symptom Cough Medicine by Richardson-Vicks, Inc., Vicks NyQuil Nighttime Colds Medicine-Original & Cherry Flavor by Richardson-Vicks, Inc., Vicks Pediatric Formula 44 Cough Medicine by Richardson-Vicks, Inc., Vicks Pediatric Formula 44 Cough & Colds Medicine by Richardson-Vicks, Inc., Vicks Pediatric Formula 44 Cough & Congestion Medicine by Richardson-Vicks, Inc., Ambenyl-D Decongestant Cough Formula by Forest Pharmaceuticals, Bromarest DX Cough Syrup by Warner Chilcott, BromFed-DM Cough Syrup by Muro, Codimal DM by Central Pharmaceuticals, Dimetane-DX Cough Syrup by Robins, Guaifenesin w/D-Methorphan Hydrobromide Syrup by Lederle, Humibid DM Tablets by Adams, IoTuss-DM Liquid by Muro, Medi-Tuss DM by Warner Chilcott, Phenergan with Dextromethorphan by Wyeth-Ayerst, Poly-Histine DM Syrup by Bock, Quelidrine Syrup by Abbott, Rondec-DM Oral Drops by Ross, Rondec DM Syrup by Ross, Tusibron-DM by RAM Laboratories, Tussar DM by Rorer Pharmaceuticals, and Tussi-Organidin DM Liquid by Wallace.

Delsym Cough Suppressant Syrup by McNeil Consumer contains dextromethorphan polistirex as an antitussive agent. It is believed that all of the above commercial products containing dextromethorphan are included in compositions at about neutral pH or lower.

Dextromethorphan Hydrobromide was approved in 1957 by the United States Food and Drug Administration, and this salt is being commercialized throughout the world due to manufacturing convenience. The salt is sparingly soluble below 20° C. (about 1.5 weight percent at 20° C.), while its solubility is high at higher temperature (about 25 wt. % at 85° C.), which makes it easy for crystallization.

Bromide presents a significant health risk and is the principal limiting ingredient in antitussive over-the-counter medications. Since bromide was first introduced as a medicine, multiple clinical symptoms of bromide intoxication have been reported. Large doses of bromide cause nausea and vomiting, abdominal pain, coma and paralysis. The chronic state of bromide intoxication is reported as bromism. Bromide has a long half life of about 9-12 days, which can lead to excessive accumulation. The signs and symptoms are referable to the nervous system, skin, glandular secretions, and gastrointestinal tract, as disclosed by F. X. R. van Leeuwen et al. in “1983a. Toxicity of sodium bromide in rats: effects on endocrine system and reproduction,” Food Chem. Toxicol., 21(4), 383-390. Bromism has been associated with neurotoxic effects leading to restlessness, irritability, ataxia, confusion, hallucinations, somnolence, psychosis, seizures and coma. Gastrointestinal disorders include nausea, vomiting, anorexia and constipation. Dermatological effects include acneiform, pustular and erythmatous rashes, as disclosed by KR. Olson in Poisoning and Drug Overdose (4^{th} Ed.) Appleton & Lange. (1 Nov. 2003) pp. 140-141. It has been postulated that bromide ion acts directly on certain endocrine organs such as the thyroid, adrenals and testes, thereby inducing hormonal feedback alterations in the pituitary gland. See, for example, F. X. R. van Leeuwen et al., “1983b. Endocrinologisch toxiciteitsonderzoek met natriumbromide,” Verslagen adviezen en rapporten 20, 150-153; and J. G. Loeber et al., “Effect of sodium bromide on endocrine parameters in the rat as studied by immunocytochemistry and radioimmunoassay,” Food Chem. Toxicol., 21(4), 391-404.

In an experiment on the time dependency of the effect of bromide on the thyroid gland in rats, significantly decreased thyroxine concentrations were found as soon as 3 days after feeding diets containing 4800 or 19200 ppm NaBr. This decrease was observed and remained constant during an experimental period of 12 weeks, as disclosed in van Leeuwen et al., “1983a.”

In another study disclosed by B. Sangster et al., “The influence of sodium bromide in man: a study in human volunteers with special emphasis on the endocrine and the central nervous system” Food Chem. Toxicol. 1983 August; 21(4):409-19, healthy volunteers were repeatedly given sodium bromide in oral doses of 0, 4 or 9 milligrams Br/kilogram body weight/day using a double blind design. Groups of seven males received the treatment for 12 weeks and groups of seven non-pregnant females (not using oral contraceptives) over three full cycles. Special attention was paid to possible effects on the endocrine and central nervous systems. At the start and end of the study, a full medical history, the results of physical examination, haematological studies and standard clinical chemistry and urine analyses were recorded for each subject. Except for incidental nausea, no changes were observed. Mean plasma bromide concentrations at the end of treatment were 0.07, 2.14 and 4.30 millimoles/liter for males and 0.07, 3.05 and 4.93 mmol/l for females of the 0-, 4- and 9-mg Br/kg/day groups, respectively. Only in the females receiving 9 mg Br/kg/day was there a significant increase in serum thyroxine and triiodothyronine at the end of the study compared to pre-administration values, but all concentrations remained within normal limits. No changes were observed in serum concentrations of free thyroxine, thyroxine-binding globulin, cortisol, oestradiol, progesterone or testosterone, or of thyrotropin, prolactin, luteinizing hormone (LH) and follicle-stimulating hormone before or after the administration of thyrotropin-releasing hormone and LH-releasing hormone. Analysis of neurophysiological data (EEG and visual evoked response) showed shifts in the power of various spectral bands and a shift in mean frequency in the groups on 9 mg Br/kg/day. All findings were, however, within normal limits.

A limited replication study was carried out to confirm the findings in the former study. Three groups of 15 females received (double blind) doses of 0, 4 and 9 mg Br/kg/day during three menstrual cycles. After the administration period, the 45 females were observed for another three cycles. Mean plasma bromide concentrations at the end of the treatment were 0.07, 3.22 and 7.99 mmol/l, respectively. In none of the three groups were significant changes observed in the serum thyroxine concentration, free thyroxine, triiodothyronine, thyrotropin and thyroxine-binding globulin. Clinical observation did not show effects on the thyroid or on the central nervous system. Quantitative analysis of the electroencephalogram (EEG) showed only a marginal effect in females receiving 9 mg Br/kg/day. (For further details, see B. Sangster et al., 1986. Onderzoek naar de invloed van natriumbromide bij menselijke vrijwilligers: III. Rapport nr. 348301001d.d. october 1986. Rijksinstituut voor de volksgezondheid en milieuhygiene, Bilthoven, Holland).

After oral ingestion bromide is rapidly and completely absorbed in the gastrointestinal tract and distributed almost exclusively in the extracellular fluid. The similarity of bromide to chloride gives rise to an important pharmacokinetic interaction. The two ions compete for reabsorption in the kidney. High chloride reabsorption will lead to higher bromide excretion and vice versa. The biological half-life of bromide can be decreased by administration of chloride. In rats, a normal half-life of bromide of three days will increase to 25 days on a chloride-free diet.

Bromide exerts various toxicological effects in rats. At higher doses, effects on the central nervous system have been observed. In short-term toxicity studies, motor incoordination of the hind legs and inhibition of grooming were found. The main effects of bromide are on endocrine organs. It is assumed that bromide acts directly on organs such as the thyroid, adrenals and testes, thereby inducing alteration in the pituitary gland by feed-back mechanisms. The effect on the thyroid may be explained by interaction with iodide uptake, and is the most sensitive effect in animal experiments.

In a short-term toxicity study with rats at a normal chloride intake, effects have been found on most endocrine organs; while in special studies, decreased levels of a number of hormones (thyroxine, growth hormone, testosterone and corticosterone) have been observed. On the other hand, thyroid-stimulating hormone (TSH) and insulin were increased. A “No Observable Adverse Effect Level” (NOAEL), based upon all available data on the effects on the thyroid of 300 ppm sodium bromide (240 ppm bromide), equivalent to 12 mg bromide/kg/day was established.

In a reproduction study in rats, complete infertility was observed at the highest dose level of 19200 ppm sodium bromide, whereas at 4800 ppm, fertility and viability of the offspring were reduced. At 1200 ppm, no effects on reproduction were observed. The effects on fertility were reversible. Bromide was not mutagenic in the Ames test.

Heretofore, a number of patents and published patent applications have disclosed antitussive liquid compositions, the relevant portions of which may be briefly summarized as follows:

U.S. Pat. No. 4,892,877, issued to Sorrentino on Jan. 9, 1990, discloses liquid compositions for the treatment of coughs comprising both dextromethorphan and phenol, the compositions having a pH of 5-9. U.S. Pat. No. 4,427,681, issued to Munshi on Jan. 24, 1984, discloses thixotropic compositions for the treatment of coughs comprising both dextromethorphan and AVICEL® RC-591. U.S. Pat. No. 5,196,436, issued to Smith on Mar. 23, 1993, discloses antitussive pharmaceutical compositions for the peroral administration of dextromethorphan, the composition being at a pH of from about 8 to about 11. The disclosures of these United States patents are hereby incorporated by reference.

It is noted that agave was cultivated for centuries by the native Indian population for fibers, food and drinks. Agave syrup or agave nectar began appearing on health food store shelves in the early 2000s. Agave syrup, also known as agave nectar, is a sweetener commonly produced in Mexico from the Agave americana plant (also called Century Plant). Agave syrup is similar to honey in color and texture, but it is not as viscous and flows more easily. Agave nectar is available in light or dark colors, the light liquid typically having been filtered. Agave has saponins and fructans. Inulin is a type of fructan that has many health benefits. Saponins are found in many plant roots, the most famous being ginseng.

Agave nectar is obtained from the agave plant grown in arid regions, by extracting the agave juice therefrom and processing it into a syrup. See, for example, U.S. Pat. No. 5,846,333 of Partida et al., the disclosure of which is incorporated herein by reference. Commercially, other names for Agave nectar have been “Sweetener” or “Syrup” as well as other similar descriptive or extension names. However, it is generally considered a syrup-like product which can be processed as an organic, natural, or raw state. It can be light to dark in color, thicker or thinner in consistency (viscosity), and even made into powder or crystals if dehydrated totally.

U.S. Pat. Application Publication No. US 2009/0104326 of Catani discloses a solid sweetening composition having erythritol and a secondary sweetener in a single solid matrix, a method of making the solid sweetening composition and methods of sweetening a comestible. U.S. Pat. Application Publication No. US 2010/0029581 of Dhillon-Gill discloses a nutritional supplement comprising about 2 parts roasted, ground flaxseed, about 2 parts of chick pea flour, about 1.5-2 parts of whole wheat or brown rice flour, about 1 part of raw, ground almonds and optionally, about 0.5-1 part of whole wheat bran. The supplement may additionally contain one or both of raw blue agave nectar and sunflower oil. The supplement may also contain cardamom and/or ginger. The nutritional supplement comprises at least about 1.5 grams of Omega-3 fatty acids, less than about 1.5 grams (g) of glucose, and less than about 0.6 g of sucrose in a serving of about 2 ounces (about 59 ml). A method of supplementing the nutrition of an individual comprises administering the nutritional supplement to the individual.

U.S. Pat. Application Publication No. US 2009/0148580 of Heyer discloses using natural agave extract as a sweetener to replace all or part of the high-calorie sugars and/or artificial sweeteners added in foods and medicines, thereby promoting an important reduction of calories and the elimination of artificial sweeteners by using natural agave extract as the main sweetening ingredient. U.S. Pat. Application Publication No. US 2009/0029009 of Dimitri discloses an all natural beverage comprising rice syrup, agave nectar, fruit juice and electrolytes. The beverage is useful for rapid hydration and replenishment of lost carbohydrates and electrolytes after exercise, physical exertion, or exposure to heat. The disclosure of all of the preceding United States published patent applications are incorporated herein by reference.

This background information is provided for the purpose of making information believed by the applicants to be of possible relevance to the present invention. No admission is necessarily intended, nor should it be construed, that any of the preceding information constitutes prior art against the present invention.

There remains a need for an effective cough suppressant which does not have the harmful side effects of bromide and polistirex. Additionally, to the best of the applicants' knowledge, no cough suppressant has been previously produced which does not contain sodium and/or preservatives. There remains a need for a cough suppressant lacking such ingredients as well.

### SUMMARY OF THE INVENTION

Accordingly, embodiments of the present invention are provided that meet at least one or more of the following objects of the present invention.

It is an object of this invention to provide dextromethorphan compositions free of bromide, polistirex, and sodium, for oral administration which will provide a more safe antitussive action than commercially available compositions.

It is also an object of this invention to provide methods for achieving rapid antitussive action from dextromethorphan compositions free of bromide, polistirex, and sodium.

In one aspect of the invention, pharmaceutical compositions are provided for oral administration which comprise a safe and effective amount of dextromethorphan and an orally-acceptable pharmaceutical carrier. The compositions are free of bromide, polistirex, and sodium. In some embodiments, the compositions are in liquid form and have a pH of from about 3.5 to about 6.5.

In another aspect of the invention, pharmaceutical compositions are provided for oral administration, which comprise a safe and effective amount of dextromethorphan, safe and effective amounts of cough/cold drug actives, and an orally-acceptable pharmaceutical carrier which is free of bromide, polistirex, and sodium. In some embodiments, the liquid compositions have a pH of from about 3.5 to about 6.5.

In another aspect of the invention, there is provided a pharmaceutical composition, in dosage unit form, for oral administration comprising a safe and effective amount of dextromethorphan and an orally-acceptable pharmaceutical carrier comprising agave nectar, water and ethanol being at a pH of about 3 to about 6.5, wherein the pharmaceutical composition is free of bromide, sodium and polistirex. The composition may include from about 1 mg to about 50 mg dextromethorphan per dose. In some embodiments, the composition is an aqueous-based solution. The composition may be an agave-nectar based liquid.

In another aspect of the invention, there is provided a method of treating or preventing cough in humans by orally administering to the human a safe and effective amount of a composition of the present invention.

### BRIEF DESCRIPTION OF THE FIGURES

The following drawing forms part of the present specification and is included to further demonstrate certain aspects of the present invention. The invention may be better understood by reference to this drawing in combination with the detailed description of specific embodiments presented herein.

FIG. 1. The chemical structure of dextromethorphan.

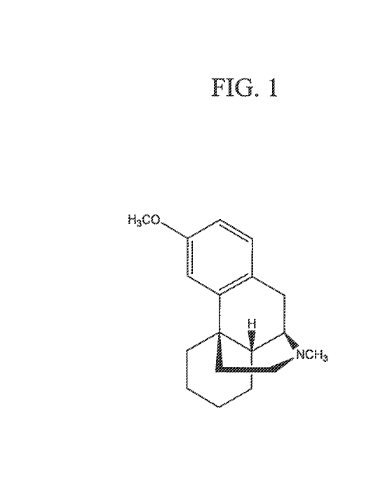

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, and alterations and modifications of the compositions and methods herein and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”

As used herein, the term “about” refers to a ±10% variation from the nominal value. It is to be understood that such a variation is always included in any given value provided herein, whether or not it is specifically referred to.

The compositions and methods of the present invention comprise a safe and effective amount of dextromethorphan, which can include pharmaceutically acceptable salts thereof. In some embodiments, the compositions comprise one or more other drug actives. The phrase “safe and effective amount”, as used herein, means an amount of drug active high enough to provide a significant positive modification of the condition to be treated, but low enough to avoid serious side effects (at a reasonable benefit/risk ratio), within the scope of sound medical judgment. A safe and effective amount of drug active will vary with the particular condition being treated, the age and physical condition of the patient being treated, the severity of the condition, the duration of the treatment, the nature of concurrent therapy and like factors.

Dextromethorphan is known to have pharmacological activity as an antitussive agent. As used herein, “dextromethorphan” means racemethorphan, 3-methoxy-17-methylmorphinan; also known as (d1-cis-1,3,4,9,10,10a-hexahydro-6-methoxy-11-methyl-2H-10,4a-iminoethanophenanthrene; also known per IUPAC nomenclature as (+)-3-methoxy-17-methyl-(9α,13α,14α)-morphinan.

In some embodiments, the pharmaceutically acceptable salt of dextromethorphan is the hydrochloride salt.

In some embodiments, the compositions of the present invention comprise from about 1 milligram (mg) to about 50 mg dextromethorphan per dose. In some embodiments, the compositions comprise from about 2.5 mg to about 30 mg dextromethorphan per dose. In some embodiments, the compositions are liquid compositions. In some embodiments, the liquid compositions comprise from about 0.02% to about 1.5% dextromethorphan, from about 0.05% to about 1% dextromethorphan, or from about 0.1% to about 0.3% dextromethorphan by weight. In some embodiments, a typical dose for a liquid antitussive composition is from about 1 milliliter (ml) to about 30 ml. In some embodiments, a dose of liquid cough syrup is more typically from about 5 ml to about 20 ml, especially about 15 ml.

In some embodiments, compositions of the present invention comprise a safe and effective amount of dextromethorphan, and an orally-acceptable pharmaceutical carrier. In some embodiments, the compositions are free of bromide, polistirex, and sodium, and have a pH of from about 3 to about 6.5, preferably of from about 3.5 to about 6.5, more preferably still of from about 4.2 to about 6, and most preferably of about 5.5. Other preferred compositions of the present invention comprise a safe and effective amount of dextromethorphan, safe and effective amounts of other cough/cold drug actives, and an orally-acceptable pharmaceutical carrier, the compositions being free of bromide, polistirex, and sodium and having a pH of from about 3 to about 6.5, preferably of from about 3.5 to about 6.5, more preferably still of from about 4.2 to about 6, and most preferably of about 5.5.

It has been found that the compositions of the present invention result in faster attainment of therapeutic blood levels of dextromethorphan, maintenance of such therapeutic blood levels for a longer time, and/or higher peak blood levels of dextromethorphan, without the side effects of bromide, polistirex, and sodium.

The compositions of the present invention preferably have an acidic buffering strength sufficient to keep dextromethorphan and other drug actives in solution without the presence of sodium.

The compositions of the present invention comprise a pharmaceutically-acceptable carrier preferably comprising a high fructose syrup such as high fructose corn syrup and agave nectar.

The compositions of the present invention are intended for oral administration. Examples of such compositions include preferred liquid compositions, especially aqueous-based liquid compositions, such as syrups, elixirs, suspensions, sprays, and drops.

Dextromethorphan hydrochloride is highly soluble in water at the pH of the compositions of the present invention. However, the compositions of the invention may contain sufficient levels of one or more cosolvents. Preferred cosolvents for this purpose include ethanol, propylene glycol, polyethylene glycol, glycerin, and sorbitol; more preferred cosolvents include ethanol, propylene glycol and glycerin.

For the liquid compositions of the present invention, the carrier preferably includes at least one of the following ingredients: sweetening agents, such as sucrose, corn syrup, agave nectar, invert sugar, dextrose, aspartame, sorbitol, honey, and magnasweet; and other flavoring agents. The preferred carrier for the invention includes agave nectar.

The compositions of the present invention also may comprise one or more other active drug agents useful for treating coughs and/or colds (hereinafter cough/cold drug actives). Cough/cold drug actives commonly combined with antitussive agents in commercial products are preferred. Cough/cold drug actives useful in the compositions of the present invention include antihistamines, bronchodilators, decongestants, expectorants, local anesthetics and anti-inflammatory/analgesics. Preferred examples of such optional drug actives and preferred amounts per unit dose in the compositions of the present invention include the following: antihistamines, such as chlorpheniramine (preferably from about 1 mg to about 8 mg, more preferably from about 2 mg to about 4 mg) and its salts (e.g., maleate); diphenhydramine (preferably from about 6 mg to about 50 mg, more preferably from about 12 mg to about 25 mg) and its salts (e.g., hydrochloride); brompheniramine (preferably from about 1 mg to about 8 mg, more preferably from about 2 mg to about 4 mg) and its salts; doxylamine (preferably from about 2 mg to about 20 mg, more preferably from about 6 mg to about 12 mg) and its salts (e.g., succinate); triprolidine (preferably from about 0.5 mg to about 4 mg, more preferably from about 1 mg to about 3 mg) and its salts (e.g., hydrochloride); bronchodilators, such as ephedrine (preferably from about 5 mg to about 50 mg, more preferably from about 10 mg to about 25 mg) and its salts (e.g., hydrochloride, sulfate); decongestants, such as pseudoephedrine (preferably from about 10 mg to about 100 mg, more preferably from about 30 mg to about 60 mg) and its salts (e.g., hydrochloride); phenylephrine (preferably from about 2 mg to about 20 mg, more preferably from about 5 mg to about 10 mg) and its salts (e.g., hydrochloride); phenylpropanolamine (preferably from about 5 mg to about 50 mg, more preferably from about 12 mg to about 25 mg) and its salts (e.g., hydrochloride); expectorants, such as guaifenesin (preferably from about 50 mg to about 400 mg, more preferably from about 100 mg to about 200 mg); local anesthetics, such as benzocaine, (preferably from about 1 mg to about 25 mg, more preferably from about 2 mg to about 15 mg); phenol (preferably from about 10 mg to about 150 mg, more preferably from about 20 mg to about 50 mg); dyclonine (preferably from about 1 mg to about 10 mg, more preferably from about 2 mg to about 4 mg) and its salts (e.g., hydrochloride); lidocaine (preferably from about 2 mg to about 20 mg, more preferably from about 4 mg to about 10 mg) and its salts (e.g., hydrochloride); butacaine (preferably from about 5 mg to about 50 mg, more preferably from about 10 mg to about 20 mg) and its salts (e.g. sulfate, hydrochloride); benzyl alcohol (preferably from about 50 mg to about 750 mg, more preferably from about 100 mg to about 500 mg); dibucaine (preferably from about 0.1 mg to about 4 mg, more preferably from about 0.5 mg to about 2 mg) and its salts (e.g., hydrochloride); tetracaine (preferably from about 0.1 mg to about 4 mg, more preferably from about 0.5 mg to about 2 mg) and its salts (e.g., hydrochloride); phenolate sodium (preferably from about 10 mg to about 150 mg, more preferably from about 20 mg to about 50 mg); salicyl alcohol (preferably from about 20 mg to about 200 mg, more preferably from about 50 mg to about 100 mg); hexylresorcinol (preferably from about 1 mg to about 10 mg, more preferably from about 2 mg to about 4 mg); menthol (preferably from about 2 mg to about 50 mg, more preferably from about 5 mg to about 25 mg); anti-inflammatory/analgesics, such as acetaminophen (preferably from about 60 mg to about 1000 mg, more preferably from about 300 mg to about 650 mg); ibuprofen (preferably from about 100 mg to about 800 mg, more preferably from about 200 mg to about 400 mg) and its salts (e.g., sodium); aspirin (preferably from about 75 mg to about 1000 mg, more preferably from about 300 mg to about 650 mg) and its salts (e.g., sodium); and naproxen (preferably from about 75 mg to about 500 mg, more preferably from about 125 mg to about 300 mg) and its salts (e.g., sodium).

In some embodiments, the antitussive composition is a liquid composition comprising dextromethorphan hydrochloride monohydrate and agave nectar. In some embodiments, the antitussive composition is administered in a dose of from about 2.5 ml to about 7.5 ml, and comprises from about 18 mg to about 50 mg of dextromethorphan hydrochloride monohydrate and from about 2 ml to about 7 ml of agave nectar. In some embodiments, the liquid composition is administered in a dose of about 5 ml and comprises about 36 mg of dextromethorphan hydrochloride monohydrate and about 4.5 ml of agave nectar. In some embodiments, the liquid composition is administered in a dose of about 5 ml and comprises about 18 mg of dextromethorphan hydrochloride monohydrate and about 4.5 ml of agave nectar. In some embodiments, the composition further comprises a cosolvent, such as ethanol, and one or more sweeteners. In some embodiments, the cosolvent is ethanol and is added in amounts of from about 0.1 ml to about 0.5 ml. In some embodiments, the sweetener is aspartame and is added in amounts of from about 2 mg to about 8 mg, from about 3 mg to about 6 mg or about 5.8 mg. In some embodiments, one or more colorants can be added. In some embodiments, the colorant is FD&C Red #40 and is added in amounts of from about 0.5 mg to about 2.0 mg.

In some embodiments, the antitussive composition is a liquid composition administered in a dose of from about 2.5 ml to about 7.5 ml, and comprising from about 10 mg to about 24 mg of dextromethorphan hydrochloride monohydrate and from about 2 ml to about 6 ml of agave nectar. In some embodiments, the composition further comprises one or more of the following: about 0.2 grams to about 0.8 grams of propylene glycol; about 0.1 ml to about 0.5 ml ethanol (99%); about 0.02 ml to about 0.07 ml of one or more flavorants; about 0.25 mg to about 2.0 mg polysorbate 80; about 50 mg to about 200 mg glycerin; about 50 mg to about 200 mg sorbitol; about 5 mg to about 20 mg aspartame; and about 0.25 mg to about 2 mg of a colorant, such as FD&C Red #40.

In some embodiments, the composition is a liquid cold and flu composition comprising dextromethorphan hydrochloride monohydrate, acetaminophen, doxylamine succinate and agave nectar. In some embodiments, the composition is administered in a dose of from about 7.5 ml to about 30 ml, and comprises from about 9 mg to about 36 mg of dextromethorphan hydrochloride monohydrate, from about 150 mg to about 650 mg of acetaminophen, from about 3 mg to about 12.5 mg doxylamine succinate, and from about 5.5 ml to about 22 ml of agave nectar. In some embodiments, the composition further comprises a cosolvent, such as ethanol, and one or more sweeteners. In some embodiments, the sweetener is aspartame and is added in amounts of from about 1 mg to about 30 mg, from about 3 to about 30 mg or about 23.2 mg. In some embodiments, the liquid composition is administered at a dose of about 15 ml and comprises about 18 mg of dextromethorphan hydrochloride monohydrate, about 325 mg acetaminophen, about 6.25 mg doxylamine succinate, and about 11 ml of agave nectar. In some embodiments, the composition further comprises one or more of the following: about 350 mg to about 1500 mg of propylene glycol; about 0.5 ml to about 3 ml ethanol (99%); about 350 mg to about 1500 mg polyethylene glycol; about 10 mg to about 30 mg of aspartame; and about 2.5 mg to about 10 mg of a colorant, such as FD&C Red #40.

In some embodiments, the composition is a liquid cold and flu composition comprising dextromethorphan hydrochloride monohydrate, acetaminophen and phenylephrine hydrochloride. In some embodiments, the composition is administered in a dose of from about 7.5 ml to about 30 ml, and comprises from about 9 mg to about 36 mg of dextromethorphan hydrochloride monohydrate, from about 50 mg to about 200 mg of acetaminophen, and from about 3 mg to about 300 mg phenylephrine hydrochloride. In some embodiments, the composition further comprises a cosolvent, such as ethanol, and one or more sweeteners or flavorants. In some embodiments, the sweetener is aspartame and is added in amounts of from about 10 mg to about 50 mg, from about 15 to about 30 mg or about 23.2 mg. In some embodiments, the liquid composition is administered in a dose of about 15 ml and comprises about 18 mg of dextromethorphan hydrochloride monohydrate, about 100 mg acetaminophen, and about 5.0 mg phenylephrine hydrochloride. In some embodiments, the composition further comprises one or more of the following: about 350 mg to about 1500 mg of propylene glycol; about 0.5 ml to about 3 ml ethanol (99%); about 350 mg to about 1500 mg polyethylene glycol; about 10 mg to about 50 mg of aspartame; about 0.025 ml to about 0.10 ml of one or more flavorants; and about 2.5 mg to about 10 mg of a colorant, such as FD&C Red #40.

In some embodiments, the composition is a liquid cough composition comprising dextromethorphan hydrochloride monohydrate, guaifenesin, phenylephrine hydrochloride, and agave nectar. In some embodiments, the composition is administered in a dose of from about 7.5 ml to about 30 ml, and comprises from about 9 mg to about 36 mg of dextromethorphan hydrochloride monohydrate, from about 50 mg to about 200 mg of guaifenesin, from about 2.5 mg to about 10 mg phenylephrine hydrochloride, and from about 5.5 ml to about 22 ml of agave nectar. In some embodiments, the composition further comprises a cosolvent, such as ethanol, and one or more sweeteners or flavorants. In some embodiments, the sweetener is aspartame and is added in amounts of from about 10 mg to about 50 mg, from about 15 to about 30 mg or about 23.2 mg. In some embodiments, the liquid composition administered in a dose of about 15 ml and comprises about 18 mg of dextromethorphan hydrochloride monohydrate, about 100 mg guaifenesin, about 5.0 mg phenylephrine hydrochloride and about 11 ml of agave nectar. In some embodiments, the composition further comprises one or more of the following: about 850 mg to about 3500 mg of propylene glycol; about 0.5 ml to about 3 ml ethanol (99%); about 0.025 ml to about 0.10 ml of one or more flavorants; about 2.5 mg to about 10 mg of a colorant, such as FD&C Red #40; about 375 mg to about 1500 mg glycerin; and about 10 mg to about 50 mg of aspartame.

In some embodiments, the composition is a liquid cough composition comprising dextromethorphan hydrochloride monohydrate, guaifenesin and agave nectar. In some embodiments, the composition is administered in a dose of from about 7.5 ml to about 30 ml, and comprises from about 9 mg to about 36 mg of dextromethorphan hydrochloride monohydrate, from about 50 mg to about 200 mg of guaifenesin, and from about 5.5 ml to about 22 ml of agave nectar. In some embodiments, the liquid composition is administered in a dose of about 15 ml and comprises about 36 mg of dextromethorphan hydrochloride monohydrate, about 200 mg guaifenesin and about 11 ml of agave nectar. In some embodiments, the composition further comprises one or more of the following: about 850 mg to about 3500 mg of propylene glycol; about 0.5 ml to about 3 ml ethanol (99%); about 0.025 ml to about 0.10 ml of one or more flavorants; about 2.5 mg to about 10 mg of a colorant, such as FD&C Red #40; about 375 mg to about 1500 mg glycerin; and about 10 mg to about 50 mg of aspartame.

In some embodiments, the composition is a liquid cough composition comprising dextromethorphan hydrochloride monohydrate, acetaminophen, chlorpheniramine maleate, phenylephrine hydrochloride and agave nectar. In some embodiments, the composition is administered in a dose of from about 7.5 ml to about 30 ml, and comprises from about 4.5 mg to about 18 mg of dextromethorphan hydrochloride monohydrate, from about 80 mg to about 320 mg acetaminophen, about 0.5 mg to about 2.0 mg chlorpheniramine maleate, from about 1.25 mg to about 5 mg phenylephrine hydrochloride and from about 5.5 ml to about 22 ml of agave nectar. In some embodiments, the liquid composition is administered in a dose of about 15 ml and comprises about 9 mg of dextromethorphan hydrochloride monohydrate, about 160 mg acetaminophen, about 1.0 mg chlorpheniramine maleate, about 2.5 mg phenylephrine hydrochloride and about 11 ml of agave nectar. In some embodiments, the composition further comprises one or more of the following: about 850 mg to about 3500 mg of propylene glycol; about 0.5 ml to about 3 ml ethanol (99%); about 0.025 ml to about 0.10 ml of one or more flavorants; about 2.5 mg to about 10 mg of a colorant, such as FD&C Red #40; about 375 mg to about 1500 mg glycerin; and about 10 mg to about 50 mg of aspartame.

In some embodiments, the composition is a solid cough composition formulated as a capsule comprising dextromethorphan hydrochloride monohydrate. In some embodiments, the capsule comprises about 9 mg to about 36 mg of dextromethorphan hydrochloride monohydrate. In some embodiments, the capsule is a 150 mg capsule and further comprises about 10 mg to about 20 mg microcrystalline cellulose, about 1 mg to about 5 mg silicon dioxide, about 1 mg to about 5 mg sodium lauryl sulfate, and about 0.5 mg to about 5 mg magnesium stearate, and about 100 mg to about 120 mg ascorbyl palmitate.

In some embodiments, the capsule is about a 140 mg capsule and comprises form about 9 mg to about 36 mg of dextromethorphan hydrochloride monohydrate and one or more of the following additional ingredients: from about 10 mg to about 15 mg microcrystalline cellulose, about 1 mg to about 5 mg silicon dioxide, about 1 mg to about 5 mg sodium lauryl sulfate, about 0.5 mg to about 5 mg magnesium stearate, about 35-60 mg magnesium sulfate, about 10 mg to about 30 mg quercetin 2H₂O, about 5 mg to about 20 mg hesperidin and about 10 mg to about 25 mg resveratrol.

In some embodiments, the capsule is about a 140 mg capsule (for cough and colds) and comprises from about 5 to about 15 mg of dextromethorphan hydrochloride monohydrate, about 1 mg to about 5 mg doxylamine succinate, and about 1 mg to about 5 mg phenylephrene HCl and one or more of the following additional ingredients: from about 50 mg to about 100 mg microcrystalline cellulose, about 1 mg to about 5 mg silicon dioxide, about 1 mg to about 5 mg sodium lauryl sulfate, about 0.5 mg to about 2 mg magnesium stearate, and about 10 mg to about 30 mg magnesium sulfate.

In some embodiments, the capsule is about a 240 mg capsule (for coughs, colds and fevers) and comprises from about 5 to about 20 mg of dextromethorphan hydrochloride monohydrate, about 80 mg to about 325 mg acetaminophen, about 1 mg to about 5 mg doxylamine succinate, and about 1 mg to about 10 mg phenylephrene HCl and one or more of the following additional ingredients: from about 15 mg to about 50 mg microcrystalline cellulose, about 1 mg to about 10 mg silicon dioxide, about 0.25 mg to about 5 mg sodium lauryl sulfate, about 0.5 mg to about 2 mg magnesium stearate, and about 10 mg to about 30 mg magnesium sulfate.

The present invention also includes methods for treating or preventing cough in humans or lower animals by orally administering a composition disclosed hereinabove. In the methods of the present invention, the daily dosage of dextromethorphan is preferably from about 0.1 mg/kg to about 10 mg/kg of body weight, more preferably from about 0.5 mg/kg to about 5 mg/kg, more preferably still from about 1 mg/kg to about 3 mg/kg. In the methods of the present invention, it is preferred that a dextromethorphan composition be orally administered to a patient from about 1 to about 10 times daily, more preferably from about 2 to about 8 times daily, more preferably still from about 3 to about 6 times daily.

### EXAMPLES

The following non-limiting examples are provided in order to illustrate the compositions and methods of the present invention. The liquid and lozenge compositions are made by conventional processes.

### Example I

### Liquid Cough Composition Ingredients Amount/5 ml Dose

| | | |
|---|---|---|
| Dextromethorphan Hydrochloride Monohydrate | 36.0 | mg |
| Agave Nectar | 4.5 | ml |
| Ethanol (99%) | 0.25 | ml |
| Flavorants | 0.02 | ml |
| Aspartame | 5.8 | mg |
| Water, Purified q.s. | | |

### Example II

### Liquid Cough Composition Ingredients Amount/5 ml Dose

| | | |
|---|---|---|
| Dextromethorphan Hydrochloride Monohydrate | 36.0 | mg |
| Agave Nectar | 4.5 | ml |
| Ethanol (99%) | 0.25 | ml |
| Flavorants | 0.02 | ml |
| Aspartame | 5.8 | mg |
| FD & C Red #40 (dye) | 1.0 | mg |
| Water, Purified q.s. | | |

### Example III

### Liquid Cough Composition Ingredients Amount/5 ml Dose

| | | |
|---|---|---|
| Dextromethorphan Hydrochloride Monohydrate | 18.0 | mg |
| Agave Nectar | 4.5 | ml |
| Ethanol (99%) | 0.25 | ml |
| Flavorants | 0.02 | ml |
| Aspartame | 5.8 | mg |
| Water, Purified q.s. | | |

A typical manufacturing process for making the above liquid cough composition (I-III) is to prepare a separate liquid phase by mixing together the following ingredients: (1) dextromethorphan hydrochloride monohydrate, ethanol, flavorants, colorant, aspartame and water. The liquid phase is then blended together with the agave nectar to produce the liquid cough composition.

### Example IV

### Liquid Cough Composition Ingredients Amount/5 ml Dose

| | | |
|---|---|---|
| Dextromethorphan Hydrochloride Monohydrate | 18.0 | mg |
| Agave nectar | 4.0 | ml |
| Propylene Glycol | 0.58 | g |
| Ethanol (99%) | 0.25 | ml |
| Flavorants | 0.02 | ml |
| Polysorbate 80 | 1.0 | mg |
| Glycerin | 100.0 | mg |
| Sorbitol | 100.0 | mg |
| Aspartame | 5.8 | mg |
| FD & C Red #40 | 1.0 | mg |
| Water, Purified q.s. | | |

### Example V

### Liquid Cold & Flu Composition Ingredients Amount/15 ml Dose

| | | |
|---|---|---|
| Dextromethorphan Hydrochloride Monohydrate | 18.0 | mg |
| Acetaminophen | 325 | mg |
| Doxylamine Succinate | 6.25 | mg |
| Agave nectar | 11.0 | ml |
| Propylene Glycol | 777.0 | mg |
| Flavorants | 0.5 | ml |
| Ethanol (95%) | 1.5 | ml |
| Polyethylene Glycol (Carbowax 400) | 750.0 | mg |
| Aspartame | 23.3 | mg |
| FD & C Red #40 | 5.1 | mg |
| Water, Purified q.s. | | |

### Example VI

### Liquid Cold & Flu Composition Ingredients Amount/15 ml Dose

| | | |
|---|---|---|
| Dextromethorphan Hydrochloride Monohydrate | 18.0 | mg |
| Acetaminophen | 100 | mg |
| Phenylephrine hydrochloride | 5.0 | mg |
| Propylene Glycol | 777.0 | mg |
| Ethanol (95%) | 1.5 | ml |
| Polyethylene Glycol (Carbowax 400) | 750.0 | mg |
| Aspartame | 23.2 | mg |
| Flavorants | 0.05 | ml |
| F, D & C Red #40 | 5.1 | mg |
| Water, Purified q.s. | | |

### Example VII

### Liquid Cough Composition Ingredients Amount/15 ml Dose

| | | |
|---|---|---|
| Dextromethorphan Hydrochloride monohydrate | 18.0 | mg |
| Guaifenesin | 100 | mg |
| Phenylephrine Hydrochloride | 5.0 | mg |
| Agave nectar | 11.0 | ml |
| Propylene Glycol | 1.74 | g |
| Ethanol (95%) | 1.5 | ml |
| Flavorants | 0.05 | ml |
| F, D & C Red #40 | 5.1 | mg |
| Glycerin | 750.0 | mg |
| Aspartame | 23.2 | mg |
| Water, Purified q.s. | | |

### Example VIII

### Liquid Cough Composition Ingredients Amount/15 ml Dose

| | | |
|---|---|---|
| Dextromethorphan Hydrochloride monohydrate | 18.0 | mg |
| Guaifenesin | 100 | mg |
| Agave nectar | 11.0 | ml |
| Propylene Glycol | 1.74 | g |
| Ethanol (95%) | 1.5 | ml |
| Flavorants | 0.05 | ml |
| F, D & C Red #40 | 5.1 | mg |
| Glycerin | 750.0 | mg |
| Aspartame | 23.2 | mg |
| Water, Purified q.s. | | |

### Example IX

### Liquid Cough Composition Ingredients Amount/15 ml Dose

| Acetaminophen | 160.0 | |
|---|---|---|
| Dextromethorphan Hydrochloride monohydrate | 9.0 | mg |
| Chlorpheniramine maleate | 1.0 | mg |
| Phenylephrine Hydrochloride | 2.5 | mg |
| Agave nectar | 11.0 | ml |
| Propylene Glycol | 1.74 | g |
| Ethanol (95%) | 1.5 | ml |
| Flavorants | 0.05 | ml |
| F, D & C Red #40 | 5.1 | mg |
| Glycerin | 750.0 | mg |
| Aspartame | 23.2 | mg |
| Water, Purified q.s. | | |

The liquid of Examples IV-IX are made by adding the active ingredients to the propylene glycol, polyethylene glycol, alcohol, flavorants, and glycerin with stirring. The solution is added to agave nectar with stirring. The dye is added to the solution with stirring. Purified water is added to volume with stirring.

The following solid compositions were prepared by weighing the required amount for 1000 capsules and the ingredients were then mixed in a high shear mixture to obtain uniformly distributed powder. The powder is then filled in 100 capsules at a time using a manual capsule filling machine. The size of the capsule was chosen according to the content per capsule.

### Example X

### Solid Cough Composition for Oral Administration

| No | Ingredient | Per Capsule (mg) |
|---|---|---|
| 1 | Dextromethorphan HCl•H2O | 18.00 |
| 2 | Microcrystalline Cellulose | 13.10 |
| 3 | Silicon Dioxide | 2.50 |
| 4 | Sodium Lauryl Sulfate | 2.50 |
| 5 | Magnesium Stearate | 1.50 |
| 6 | Ascorbyl Palmitate | 113.00 |
| | Total | 150.60 |

### Example XI

### Solid Cough Composition for Oral Administration Containing Antioxidants

| No | Ingredient | Per Capsule (mg) |
|---|---|---|
| 1 | Dextromethorphan HCl•H2O | 18.00 |
| 2 | Microcrystalline Cellulose | 13.50 |
| 3 | Silicon Dioxide | 4.10 |
| 4 | Sodium Lauryl Sulfate | 1.00 |
| 5 | Magnesium Stearate | 1.00 |
| 6 | Magnesium Sulfate | 50.00 |
| 7 | Quercetin 2H2O | 22.40 |
| 8 | Hesperidin | 10.00 |
| 9 | Resveratrol | 20.00 |
| | Total | 140.00 |

### Example XII

### Solid Cough and Cold Composition for Oral Administration

| No | Ingredient | Per Capsule (mg) |
|---|---|---|
| 1 | Dextromethorphan HCl•H2O | 9.00 |
| 2 | Doxylamine Succinate | 3.25 |
| 3 | Phenylephrene HCl | 5.00 |
| 4 | Microcrystalline Cellulose | 73.65 |
| 5 | Silicon Dioxide | 4.10 |
| 6 | Sodium Lauryl Sulfate | 1.00 |
| 7 | Magnesium Stearate | 1.00 |
| 8 | Magnesium Sulfate | 25.00 |
| | Total | 140.00 |

### Example XIII

### Solid Cough, Cold and Fever Composition for Oral Administration

| No | Ingredient | Per Capsule (mg) |
|---|---|---|
| 1 | Dextromethorphan HCl•H2O | 9.00 |
| 2 | Acetaminophen | 160.00 |
| 3 | Doxylamine Succinate | 3.25 |
| 4 | Phenylephrene HCl | 5.00 |
| 5 | Microcrystalline Cellulose | 31.65 |
| 6 | Silicon Dioxide | 4.10 |
| 7 | Sodium Lauryl Sulfate | 1.00 |
| 8 | Magnesium Stearate | 1.00 |
| 9 | Magnesium Sulfate | 25.00 |
| | Total | 240.00 |

It is, therefore, apparent that there has been provided, in accordance with the present invention, antitussive compositions containing dextromethorphan that are essentially free of bromide, sodium and polistirex. Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims.

1. An antitussive composition, in dosage unit form, for oral administration comprising a safe and effective amount of dextromethorphan hydrochloride and one or more orally-acceptable pharmaceutical excipients, wherein the composition is essentially free of bromide, sodium and polistirex.

2. The composition of claim 1 wherein the composition comprises from about 1.0 mg to about 50 mg of dextromethorphan hydrochloride.

3. The composition of claim 1 wherein the composition is liquid and comprises agave-nectar, wherein the pH of the composition is about 3 to about 6.5.

4. The composition of claim 1 wherein the composition is a solid dosage form.

5. The composition of claim 1 wherein the composition further comprises from about 100 mg to about 500 mg of acetaminophen.

6. The composition of claim 1 wherein the composition further comprises an antioxidant selected from the group consisting of quercetin, hesperidin, resveratrol, myricetin and combinations thereof.

7. The composition of claim 1 wherein the composition further comprises from about 50 mg to about 200 mg of guaifene sin.

8. The composition of claim 1 wherein the composition further comprises from about 1 mg to about 5 mg of chiorpheniramine maleate.

9. The composition of claim 1 wherein the composition further comprises from about 2 mg to about 10 mg of phenylephrine hydrochloride.

10. The composition of claim 1, wherein the composition further comprises about 2 mg to about 10 mg of doxylamine succinate.

11. The composition of claim 1, wherein the composition is at a pH of about 3.4 to about 5.5 and comprises about 2.5 mg to about 30 mg of dextromethorphan per dose.

12. A method of treating or preventing cough in a human in need thereof, comprising orally administering to the human a safe and effective amount of the composition of claim 1.
